(19) United States
(12) Patent Application Publication
(10) Pub. No.: US 2015/0203543 A1
(43) Pub. Date: Jul. 23, 2015

(54) STABILISED PROTEINS FOR IMMUNISING AGAINST STAPHYLOCOCCUS AUREUS

(71) Applicant: NOVARTIS AG, Basel (CH)

(72) Inventors: Fabio Bagnoli, Monteriggioni (IT); Fabiana Falugi, Chicago, IL (US); Guido Grandi, Segrate (IT); Massimo Mariani, Siena (IT); Mikkel Nissum, Casciano di Murlo (IT); Michele Pallaoro, Siena (IT); Silvana Savino, Tavarnelle Val di Pesa (IT)

(21) Appl. No.: 14/420,834

(22) PCT Filed: Aug. 29, 2013
(86) PCT No.: PCT/EP2013/067854

§ 371 Date: Feb. 10, 2015

Related U.S. Application Data
(60) Provisional application No. 61/695,759, filed on Aug. 31, 2012.

(51) Int. Cl. C07K 14/31; A61K 39/085

(52) U.S. Cl. CPC C07K 14/31; A61K 39/085

(57) ABSTRACT

Elimination of disulphide bond formation of cysteine-containing S. aureus antigens enhances antigen stability. The invention provides variant forms of cysteine-containing S. aureus antigen with a point mutation that replaces, deletes or modifies the cysteine residue.

This application claims the benefit of U.S. provisional application 61/695,759 filed Aug. 31, 2012, the complete contents of all of which are hereby incorporated herein by reference for all purposes.

### TECHNICAL FIELD

This invention relates to immunogenic compositions comprising antigens derived from Staphylococcus aureus and to their use in immunisation.

### BACKGROUND ART

S. aureus is a Gram-positive spherical bacterium and is the leading cause of infection of the bloodstream, lower respiratory tract, and skin and other soft tissues. It causes a range of illnesses from minor skin infections to life-threatening diseases including pneumonia and septicaemia, and the mortality associated with S. aureus per annum in the US exceeds that of any other infectious disease, including HIV/AIDS.

There is currently no authorised vaccine against S. aureus. A vaccine based on a mixture of surface polysaccharides from bacterial types 5 and 8, StaphVAX™, failed to reduce infections when compared to the placebo group in a phase III clinical trial in 2005. Reference 1 reports data on the “V710” vaccine from Merck and Intercell which is based on a single antigen, IsdB, a conserved iron-sequestering cell-surface protein [2,3]. However, the clinical trials of V710 were terminated in 2011 based on the observation that V710 was unlikely to demonstrate a statistically significant clinical benefit, and a safety concern regarding overall mortality and multi-organ dysfunction that occurred with greater frequency in vaccine recipients compared with placebo recipients [4].

Reference 5 discloses various S. aureus antigens and their combinations as vaccine strategics. Reference 6 discloses that S. aureus polypeptide antigens can be unstable in a simple buffer solution, and that antigens can be stabilised by the presence of a stabilizing additive, e.g. EDTA. Instability of the antigens is undesirable because (1) it does not allow vaccines to be stored for a long period of time before administration, and (2) inconsistency of vaccines from batch to batch can affect quality and regulatory approval requirements. Furthermore, manufacture of vaccines containing these unstable antigens can be complicated and involve multiple purification steps. Therefore it is an object of the invention to identify further strategies to stabilize S. aureus polypeptide antigens in immunogenic compositions.

### DISCLOSURE OF THE INVENTION

The inventors have found that preventing oligomerization of antigens is an effective strategy to enhance antigen stability. Various S. aureus antigens contain cysteine residues, and they can form oligomers in standard buffer solutions, including covalent dimers formed by disulphide bonds between cysteine residues. The inventors have found that compositions containing these covalent dimers can be unstable, and may form aggregates or influence the stability of the other antigens in the composition, if present. Covalent dimer formation can be prevented by replacing, modifying or deleting the cysteine residues such that disulphide bond formation is eliminated. Interestingly, preventing these antigens to form covalent dimers improves antigen stability and keeps a high total selectivity of the composition (i.e. a high proportion of single isoform relative to total antigen) and purity. Furthermore, the inventors found that these cysteine-deficient antigens remain effective in eliciting an immune response against the wild-type cysteine-containing antigens. Therefore, cysteine-deficient antigens can be included in vaccine formulations to improve antigen stability.

The Sta011 antigen naturally has a N-terminus cysteine in its mature form. Sta011 forms dimers and protein isoforms (pI at 6.0, 7.8 and 8.0) which vary in proportion from batch to batch. The inventors found that deletion of cysteine stops dimerization of a single isoform (pI 8.0), and gives a protein easier to characterise and analyse, without negatively impacting immunogenicity. Thus, the invention provides a polypeptide comprising an amino acid sequence that has at least 90% (e.g. ≧91%, ≧92%, ≧93%, ≧94%, ≧95%, ≧96%, ≧97%, ≧98%, ≧99%, ≧99.5%) identity to SEQ ID NO: 7, wherein the polypeptide has no free thiol group, and can elicit antibodies (e.g. when administered to a human) which recognise a wild-type Sta011 antigen (e.g. a S. aureus protein consisting of SEQ ID NO: 5). The polypeptide cannot form covalent dimers via disulphide bonds.

The invention provides an immunogenic composition comprising a polypeptide of the invention. The composition can be in aqueous form, in which case it ideally has a pH of between 5 and 8. The composition may also include an adjuvant e.g. an aluminium salt.

In some embodiments of the invention, the immunogenic composition comprises further antigens which can be polypeptides and/or saccharides. For example, they can also include one or more S. aureus capsular saccharide conjugate(s) e.g. against a serotype 5 and/or a serotype 8 strain. In other embodiments, the composition includes no additional staphylococcal polypeptide antigens. In other embodiments, the composition includes no additional staphylococcal antigens. In yet another embodiment, the composition includes no additional antigens.

The invention also provides a lyophilizate of the immunogenic composition of the invention. This lyophilizate can be reconstituted with aqueous material to provide an aqueous immunogenic composition of the invention. For administration, the lyophilizate is thus reconstituted with a suitable liquid diluent (e.g. a buffer, saline solution, water for injections (WFI)). The liquid diluent can include an adjuvant e.g. an aluminium salt or an oil-in-water emulsion adjuvant.

Sta011

The ‘Sta011’ antigen is disclosed as a useful immunogen in Reference 5. It was originally annotated merely as ‘lipoprotein’. In the NCTC 8325 strain Sta011 is SAOUHSC_{—}00052 and has amino acid sequence SEQ ID NO: 1 (GI:88193872). The known Sta011 antigen has a N-terminus cysteine in its mature form, which may be lipidated. Wild-type cysteine-containing Sta011 can exist as a monomer or an oligomer (e.g. covalent dimer), with Ca⁺⁺ ions favouring oligomerization.

The invention uses a variant form of Sta011 that cannot form covalent dimers via disulphide bonds. The polypeptide does not contain any free thiol group (under reducing conditions). It can elicit antibodies (e.g. when administered to a human) which recognise a wild-type Sta011 antigen (e.g. SEQ ID NO: 5, 13, 14 or 15). The polypeptide may comprise an amino acid sequence having 80% or more identity (e.g. 85%, 90%, 91%, 92%, 93%, 94%, 95%, 96%, 97%, 98%, 99%, 99.5% or more) to any of SEQ ID NOs: 7-12.

SEQ ID NO: 7 is amino acid residues 26-256 of SEQ ID NO: 1. Compared to SEQ ID NO: 7, SEQ ID NO: 8 has an additional amino acid residue ‘X’ at the N-terminus, wherein ‘X’ is an amino acid that does not contain a free thiol group (e.g. is Ser=SEQ ID NO: 12). SEQ ID NO: 9 has a Met-Gly-sequence at the N-terminus of SEQ ID NO: 7. SEQ ID NO: 10 has a Met-Gly-sequence at the N-terminus of SEQ ID NO: 8. SEQ ID NO: 11 has the sequence of SEQ ID NO: 10, wherein ‘X’ is serine. A Sta011 polypeptide comprising any of SEQ ID NOs: 7-12 can be used with the invention.

Variant forms of SEQ ID NO: 1 which may be used for preparing a Sta011 polypeptide of the invention include, but are not limited to, SEQ ID NOs: 2, 3 and 4 with various Ile/Val/Leu substitutions. Compared to SEQ ID NO: 1, SEQ ID NO: 2 has Leu-146 instead of Ile-146 and Ile-165 instead of Leu-165. Compared to SEQ ID NO: 1, SEQ ID NO: 3 has Val-146 instead of Ile-146 and 11e165 instead of Leu-165. Compared to SEQ ID NO: 1, SEQ ID NO: 4 has Leu-146 instead of Ile-146 and Val-165 instead of Leu-165. The first 23 N-terminal amino acids of SEQ ID NOs: 1-4 (i.e. the signal peptide) can be usefully omitted to provide SEQ ID NOs: 5, 13, 14 and 15, respectively. Thus, a Sta011 polypeptide of the invention can comprise residues 26-256 of any of SEQ ID NOs: 1-4, and it can elicit antibodies (e.g. when administered to a human) which recognise the mature Sta011 antigen (e.g. SEQ ID NO: 5, 13, 14 or 15).

A useful variant form of Sta011 may comprise at least one point mutation that replaces, modifies or deletes the cysteine residue present in the wild-type form of the antigen. For example, a Sta011 polypeptide may comprise an amino acid sequence having SEQ ID NO: 6, wherein the cysteine residue at position 3 of SEQ ID NO: 6 is replaced, modified or deleted. Preferably, the replacement is with a serine residue (e.g. providing SEQ ID NO: 11) or an alanine residue. Alternatively, the cysteine residue is deleted.

Hybrid Polypeptides

Antigens used in the invention may be present in the composition as individual separate polypeptides. Where more than one antigen is used, however, they do not have to be present as separate polypeptides. Instead, at least two (e.g. 2, 3, 4, 5, or more) antigens can be expressed as a single polypeptide chain (a ‘hybrid’ polypeptide), as described in Reference 5. The hybrid polypeptide used with the invention ideally has no free thiol group (under reducing conditions).

Hybrids consisting of amino acid sequences from two, three, four, or more antigens are useful. In particular, hybrids consisting of amino acid sequences from two, three, four, or five antigens are preferred, such as two antigens.

Different hybrid polypeptides may be mixed together in a single formulation. The hybrid polypeptides can also be combined with conjugates or non-S. aureus antigens as described elsewhere herein.

Usefully, these hybrid polypeptides can elicit antibodies (e.g. when administered to a human) that recognise each of the wild-type staphylococcal proteins represented in the hybrid.

In some embodiments antigens in a single hybrid polypeptide are joined together by a linker amino acid sequence. Linker amino acid sequences will typically be short (e.g. 20 or fewer amino acids i.e. 20, 19, 18, 17, 16, 15, 14, 13, 12, 11, 10, 9, 8, 7, 6, 5, 4, 3, 2, 1). Examples comprise short peptide sequences which facilitate cloning, or poly-glycine linkers. Other suitable linker amino acid sequences will be apparent to those skilled in the art.

Polypeptides used with the Invention

The invention uses variant forms of S. aureus antigens that do not form disulphide bonds. S. aureus antigens that contain free thiol groups (e.g. cysteine amino acids) can form oligomers, including covalent homo- or hetero-dimers in standard buffers. The covalent dimers are usually produced by oxidation of the thiol groups of cysteine residues resulting in a disulphide bond (i.e. the formation of a cystine). To eliminate covalent dimer formation, the polypeptides of the invention do not contain any free thiol groups (under reducing conditions) that can react to form disulphide bonds. A free thiol group, also known as an unprotected thiol group, or a free or unprotected —SH, has a reactive sulphur atom. A cysteine amino acid residue has a free thiol group (under reducing conditions), and thus the polypeptides of the invention do not contain any cysteine amino acid residue. A cysteine residue can be derivatised such that the thiol group is protected and cannot react to form disulphide bonds, e.g. by adding a thiol protecting group. Thiol protecting groups are known in the art, e.g. thioether, thioester or derivatives thereof [7]. Thus, the polypeptides of the invention may contain derivatised cysteine amino acid residues, provided that the derivatised cysteine amino acid residues do not have free thiol groups (under reducing conditions) that can form disulphide bonds.

In some exceptional embodiments, a polypeptide can include a thiol group, but this thiol group is not part of the side chain in a cysteine residue. Ideally, however, a polypeptide includes no thiol groups at all.

Preferably the polypeptide contains neither cysteine nor cystine.

In some embodiments, the polypeptide may contain amino acid ‘X’. ‘X’ can be any amino acid, provided that it does not contain a free thiol group. The amino acid can be a natural or a non-natural amino acid. Natural amino acids are known in the art, e.g. alanine, arginine, asparagine, aspartic acid, glutamine, glutamic acid, glycine, histidine, isoleucine, leucine lysine, methionine, phenylalanine, proline, serine, threonine, tryptophan, tyrosine or valine. Cysteine has a free thiol group, and so ‘X’ cannot be a cysteine residue. A non-natural amino acid can be a derivatised or modified amino acid. ‘X’ can be a derivatised amino acid that does not contain a free thiol group, e.g. methyl-cysteine.

Polypeptides used with the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides used with the invention can be prepared by various means (e.g. recombinant expression, purification from cell culture, chemical synthesis, etc.). Recombinantly-expressed proteins are preferred, particularly for hybrid polypeptides.

Antigens in composition of the invention are separated from the organism in which they were expressed. Sta011 polypeptides are thus provided in purified or substantially purified for before being used i.e. substantially free from other staphylococcal or host cell polypeptides. A Sta011 polypeptide is generally at least about 80% pure (by weight) before being used with the invention, and usually at least about 90% pure i.e. less than about 20%, and preferably less than about 10% (e.g. <5%) of a Sta011 composition is made up of other polypeptides.

Preferred polypeptides used with the invention have a N-terminus methionine, but in some embodiments a methionine which was present at the N-terminus of a nascent polypeptide may be absent from the polypeptide in a composition of the invention.

Polypeptides used with the invention are preferably staphylococcal polypeptides.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulphide bond formation, glycosylation, lipidation, acetylation, phosphorylation, or any other manipulation or modification, such as conjugation with a labelling component. Also included are, for example, polypeptides containing one or more analogs of an amino acid (including, for example, unnatural amino acids, etc.), as well as other modifications known in the art. Polypeptides can occur as single chains or associated chains.

The invention provides polypeptides comprising a sequence —P-Q- or -Q-P—, wherein: —P— is an amino acid sequence as defined above and -Q- is not a sequence as defined above i.e. the invention provides fusion proteins, provided that the polypeptides do not contain any free thiol group. Where the N-terminus codon of —P— is not ATG, but this codon is not present at the N-terminus of a polypeptide, it will be translated as the standard amino acid for that codon rather than as a Met. Where this codon is at the N-terminus of a polypeptide, however, it will be translated as Met. Examples of -Q- moieties include, but are not limited to, histidine tags (i.e. His where n=3, 4, 5, 6, 7, 8, 9, 10 or more), maltose-binding protein, or glutathione-S-transferase (GST).

Although expression of the polypeptides of the invention may take place in a Staphylococcus, the invention will usually use a heterologous host for expression (recombinant expression). The heterologous host may be prokaryotic (e.g. a bacterium) or eukaryotic. It may be E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc. Compared to the wild-type S. aureus genes encoding polypeptides of the invention, it is helpful to change codons to optimise expression efficiency in such hosts without affecting the encoded amino acids.

Nucleic Acids

The invention provides nucleic acid encoding polypeptides and hybrid polypeptides of the invention. It also provides nucleic acid comprising a nucleotide sequence that encodes one or more polypeptides or hybrid polypeptides of the invention.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

Methods of manipulating nucleic acids and expressing the encoded proteins are known in the art, and include those described in References 41 and 65. A nucleic acid sequence may be modified by replacing the codon for cysteine with a codon for another amino acid. The cysteine may be replaced with any other amino acid, including serine, alanine, glycine, valine, leucine, or isoleucine, or modified forms of an amino acid that does not have free thiol groups (i.e. cannot readily form disulphide bonds). Alternatively, the cysteine residue may simply be deleted from the sequence. Thus, a deletion must remove the codon for the cysteine from the nucleic acid sequence without introducing a frameshift. Techniques for making substitution and deletion mutations at predetermined sites in a nucleic acid having a known sequence are well known and include, but are not limited to, primer mutagenesis and other forms of site-directed mutagenesis.

The invention also provides nucleic acid comprising nucleotide sequences having sequence identity to such nucleotide sequences. Identity between sequences is preferably determined by the Smith Waterman homology search algorithm as described above. Such nucleic acids include those using alternative codons to encode the same amino acid.

Nucleic acid according to the invention can take various forms (e.g. single stranded, double stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double stranded form. Nucleic acids of the invention are preferably provided in purified or substantially purified form i.e. substantially free from other nucleic acids (e.g. free from naturally-occurring nucleic acids), particularly from other staphylococcal or host cell nucleic acids, generally being at least about 50% pure (by weight), and usually at least about 90% pure. Nucleic acids of the invention are preferably staphylococcal nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

Strains and Variants

An exemplary amino acid and nucleotide sequence for the antigens described herein can easily be found in public sequence databases from the NCTC 8325 and/or Newman S. aureus strain using their GI numbers, for example, but the invention is not limited to sequences from the NCTC 8325 and Newman strains. Genome sequences of several other strains of S. aureus are available, including those of MRSA strains N315 and Mu50 [8], MW2, N315, COL, MRSA252, MSSA476, RF122, USA300 (very virulent), JH1 and JH9. Standard search and alignment techniques can be used to identify in any of these (or other) further genome sequences the homolog of any particular sequence from the Newman or NCTC 8325 strain. Moreover, the available sequences from the Newman and NCTC 8325 strains can be used to design primers for amplification of homologous sequences from other strains. Thus the invention is not limited to these two strains, but rather encompasses such variants and homologs from other strains of S. aureus, as well as non-natural variants. In general, suitable variants of a particular SEQ ID NO include its allelic variants, its polymorphic forms, its homologs, its orthologs, its paralogs, its mutants, etc., provided they do not contain any free thiol group.

Thus, for instance, polypeptides used with the invention may, compared to the SEQ ID NO herein, include one or more (e.g. 1, 2, 3, 4, 5, 6, 7, 8, 9, etc.) amino acid substitutions, such as conservative substitutions (i.e. substitutions of one amino acid with another which has a related side chain), provided that the new amino acid residue does not contain a free thiol group. The polypeptides of the invention do not contain any cysteine residue. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptide of the invention cannot be substituted with a cysteine. The polypeptides may also include one or more (e.g. 1, 2, 3, 4, 5, 6, 7, 8, 9, etc.) single amino acid deletions relative to the SEQ ID NO sequences. The polypeptides may also include one or more (e.g. 1, 2, 3, 4, 5, 6, 7, 8, 9, etc.) insertions (e.g. each of 1, 2, 3, 4 or 5 amino acids) relative to the SEQ ID NO sequences, provided that the inserted amino acid residue does not contain any free thiol group (e.g. the inserted amino acid is not a cysteine).

Similarly, a polypeptide used with the invention may comprise an amino acid sequence that:
is identical (i.e. 100% identical) to a sequence disclosed in the sequence listing;
shares sequence identity (e.g. 80%, 85%, 90%, 91%, 92%, 93%, 94%, 95%, 96%, 97%, 98%, 99%, 99.5% or more) with a sequence disclosed in the sequence listing;
has 1, 2, 3, 4, 5, 6, 7, 8, 9 or 10 (or more) single amino acid alterations (deletions, insertions, substitutions), which may be at separate locations or may be contiguous, as compared to the sequences of (a) or (b); and
when aligned with a particular sequence from the sequence listing using a pairwise alignment algorithm, each moving window of x amino acids from N-terminus to C-terminus (such that for an alignment that extends to p amino acids, where p>x, there are p-x+1 such windows) has at least x·y identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if x·y is not an integer then it is rounded up to the nearest integer. The preferred pairwise alignment algorithm is the Needleman-Wunsch global alignment algorithm [9], using default parameters (e.g. with Gap opening penalty=10.0, and with Gap extension penalty=0.5, using the EBLOSUM62 scoring matrix). This algorithm is conveniently implemented in the needle tool in the EMBOSS package [10];
   provided that the polypeptide does not contain any free thiol group.

Where hybrid polypeptides are used, the individual antigens within the hybrid (i.e. individual —X— moieties) may be from one or more strains. Where n=2, for instance, X₂ may be from the same strain as X₁ or from a different strain. Where n=3, the strains might be (i) X₁=X₂=X₃ (ii) X₁=X₂≠X₃ (iii) X₁≠X₂=X₃ (iv) X₁≠X₂≠X₃ or (v) X₁=X₃≠X₂, etc.

Within group (c), deletions or substitutions may be at the N-terminus and/or C-terminus, or may be between the two termini. Thus a truncation is an example of a deletion. Truncations may involve deletion of up to 40 (or more) amino acids at the N-terminus and/or C-terminus. N-terminus truncation can remove leader peptides e.g. to facilitate recombinant expression in a heterologous host. C-terminus truncation can remove anchor sequences e.g. to facilitate recombinant expression in a heterologous host.

In general, when an antigen comprises a sequence that is not identical to a complete S. aureus sequence from the sequence listing (e.g. when it comprises a sequence listing with <100% sequence identity thereto, or when it comprises a fragment thereof) it is preferred in each individual instance that the antigen can elicit an antibody which recognises the respective complete S. aureus sequence.

Combinations with Saccharides

The immunogenic compositions of the invention may further comprise saccharide antigens (e.g. known saccharide antigens include the exopolysaccharide of S. aureus, which is a poly-N-acetylglucosamine (PNAG), and the capsular saccharides of S. aureus, which can be e.g. from type 5, type 8 or type 336). In some embodiments a composition does not include a S. aureus saccharide antigen.

Combinations with Non-Staphylococcal Antigens

The immunogenic compositions of the invention may further comprise non-staphylococcal antigens, and in particular with antigens from bacteria associated with nosocomial infections. For example, the immunogenic composition may further comprise one or more antigen(s) selected from the group consisting of: Clostridium difficile; Pseudomonas aeruginosa; Candida albicans; and extraintestinal pathogenic Escherichia coli. Further suitable antigens for use in combination with staphylococcal antigens of the invention are listed on pages 33-46 of Reference 11.

Preferred Compositions

In some embodiments the composition may include one or more further polypeptides. If the composition does include one or more further polypeptides, it is preferred that these do not contain any free thiol groups. Preferably, the further polypeptides are staphylococcal polypeptides, e.g. the S. aureus polypeptides disclosed in Reference 5.

The composition of the invention is particularly useful when using TLR7 agonists of formula (K). These agonists are discussed in detail in Reference 12:

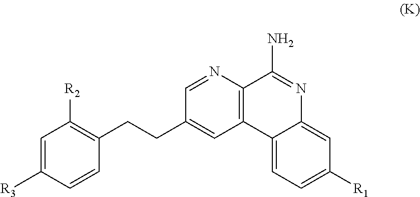

wherein:
R¹ is H, C₁-C₆alkyl, —C(R⁵)₂OH, -L¹R⁵, -L¹R⁶, -L²R⁵, -L²R⁶, —OL²R⁵, or —OL²R⁶;
L¹ is —C(O)— or —O—;
L² is C₁-C₆alkylene, C₂-C₆alkenylene, arylene, heteroarylene or —((CR⁴R⁴)ₚO)_{q}(CH₂)ₚ—, wherein the C₁-C₆alkylene and C₂-C₆alkenylene of L² are optionally substituted with 1 to 4 fluoro groups;
each L³ is independently selected from C₁-C₆alkylene and —((CR⁴R⁴)ₚO)_{q}(CH₂)ₚ—, wherein the C₁-C₆alkylene of L³ is optionally substituted with 1 to 4 fluoro groups;
L⁴ is arylene or heteroarylene;
R² is H or C₁-C₆alkyl;
R³ is selected from C₁-C₄alkyl, -L³R⁵, -L¹R⁵, -L³R⁷, -L³L⁴L³R⁷, -L³L⁴R⁵, -L³L⁴L³R⁵, —OL³R⁵, —OL³R⁷, —OL³L⁴R⁷, —OL³L⁴L³R⁷, —OR⁸, —OL³L⁴R⁵, —OL³L⁴L³R⁵ and —C(R⁵)₂OH;
each R⁴ is independently selected from H and fluoro;
R⁵ is —P(O)(OR⁹)₂,
R⁶ is —CF₂P(O)(OR⁹)₂ or —C(O)OR¹⁰;
R⁷ is —CF₂P(O)(OR⁹)₂ or —C(O)OR¹⁰;
R⁸is H or C₁-C₄alkyl;
each R⁹ is independently selected from H and C₁-C₆alkyl;
R¹⁰ is H or C₁-C₄alkyl;
each p is independently selected from 1, 2, 3, 4, 5 and 6, and
q is 1, 2, 3 or 4.

The compound of formula (K) is preferably of formula (K′):

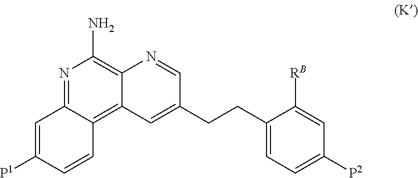

wherein:
P¹ is selected from H, C₁-C₆alkyl optionally substituted with COOH and —Y-L-X—P(O)(OR^{X})(OR^{Y});
P² is selected from H, C₁-C₆alkyl, C₁-C₆alkoxy and —Y-L-X—P(O)(OR^{X})(OR^{Y});
with the proviso that at least one of P¹ and P² is —Y-L-X—P(O)(OR^{X})(OR^{Y});
R^{B} is selected from H and C₁-C₆alkyl;
R^{X} and R^{Y} are independently selected from H and C₁-C₆alkyl;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
each p is independently selected from 1, 2, 3, 4, 5 and 6; and
q is selected from 1, 2, 3 and 4.

In some embodiments of formula (K′): P¹ is selected from C₁-C₆alkyl optionally substituted with COOH and —Y-L-X—P(O)(OR^{X})(OR^{Y}); P² is selected from C₁-C₆alkoxy and —Y-L-X—P(O)(OR^{X})(OR^{Y}); R^{B} is C₁-C₆alkyl; X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; q is selected from 1 and 2.

A preferred compound of formula (K) for use with the invention is 3-(5-amino-2-(2-methyl-4-(2-(2-(2-phosphonoethoxy)ethoxy)ethoxy)phenethyl)benzo[f][1,7]naphthyridin-8-yl)propanoic acid, or compound ‘K1’:

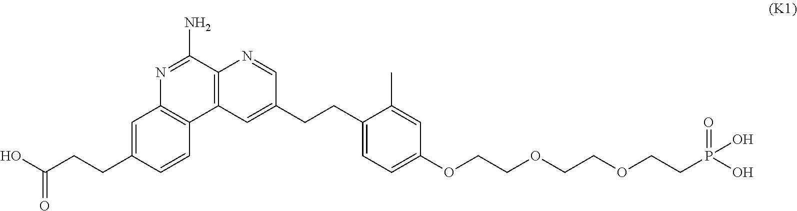 [SMILES: Cc1cc(OCCOCCOCCP(=O)(O)O)ccc1CCc1cnc2c(N)nc3cc(CCC(=O)O)ccc3c2c1]

This compound can be used as free base or in the form of a pharmaceutically acceptable salt e.g. an arginine salt.

Compounds of formula (K) can be mixed with an insoluble metal salt (preferably an aluminium salt, such as an aluminium hydroxide), and the compound is typically adsorbed to the metal salt. The Sta011 antigen (and, optionally, further antigen(s) in a composition) can also be adsorbed to the metal salt. Thus a preferred composition comprises (i) a Sta011 antigen as defined herein (ii) a TLR7 agonist of formula (K), such as formula (K1), and (iii) an insoluble metal salt, such as an aluminium hydroxide. The TLR7 agonist and the Sta011 antigen are preferably adsorbed to the metal salt.

Stabilizing Additives

In some embodiments of the invention an immunogenic composition includes a stabilizing additive. Such additives include, but are not limited to, chelators of divalent metal cations (e.g. EDTA, ethylenediaminetetraacetic acid), sugars (e.g. disaccharides such as sucrose or trehalose), sugar alcohols (e g. mannitol), free amino acids (e.g. arginine), buffer salts (e.g. phosphate, citrate), polyols (e.g. glycerol, mannitol), or protease inhibitors.

EDTA is a preferred additive. The final concentration of EDTA in the immunogenic composition of the invention can be about 1-50 mM, about 1-10 mM or about 1-5 mM, preferably about 2.5 mM.

A buffer is another useful additive, in order to control pH of a composition. This can be particularly important after reconstitution of lyophilized material. Compositions of the invention may include one or more buffer(s). Typical buffers include: a phosphate buffer; a Tris buffer; a borate buffer; a succinate buffer; a histidine buffer; or a citrate buffer. A phosphate buffer is preferable. Buffers will typically be included in the 5-20 mM range. Aqueous compositions of the invention preferably have a pH of between 5 and 8 e.g. between 5.5-6.5, or 5.9-6.1, or a pH of 6.

A saccharide or sugar alcohol (or mixture thereof e.g. a mannitol sucrose mixture) is also useful, particularly when using lyophilization. Suitable materials include, but are not limited to, mannitol, lactose, sucrose, trehalose, dextrose, etc. The use of sucrose is particularly preferred. Such materials can be present at a concentration of about 1% by weight per volume, or about 3% to about 6% by weight per volume, or up to about 10% or about 12.5% by weight per volume, preferably about 5% by weight per volume.

Lyophilization

One way of storing immunogenic compositions of the invention is in lyophilized form. This procedure can be used with or without the addition of a metal chelator (e.g. EDTA). The inventors have also shown that EDTA does not have a significant impact on the thermal characteristic of the vaccine and does not introduce any undesired plasticizing effect, thus meaning that EDTA-containing compositions can be lyophilized to further enhance storage stability.

Thus, generally, the invention also provides a lyophilizate which comprises a divalent metal cation chelator (e.g. EDTA) and at least one antigen (e.g. at least one polypeptide antigen).

The invention also provides a lyophilizate of an aqueous immunogenic composition of the invention. This is prepared by lyophilising an aqueous composition of the invention. It can then be reconstituted with aqueous material to provide an aqueous immunogenic composition of the invention. Materials present in the material which is lyophilized will remain in the lyophilizate and will thus also be present after reconstitution e.g. buffer salts, lyoprotectants (e.g. sucrose and/or mannitol), chelators, etc. If the material is reconstituted with a smaller volume of material than before lyophilization then these materials will be present in more concentrated form. The reconstituted lyophilizate preferably contains lyoprotectants (e.g. sucrose and/or mannitol) at a concentration of up to about 2.5% by weight per volume, preferably about 1% to about 2% by weight per volume. The amount of EDTA which is present in a composition prior to lyophilization is ideally at least 0.75 mM, and preferably at least 2.5 mM. A maximum of 50 mM is envisaged.

Liquid materials useful for reconstituting lyophilizates include, but are not limited to: salt solutions, such as physiological saline; buffers, such as PBS; water, such as wfi. They usefully have a pH between 4.5 and 7.5 e.g. between 6.8 and 7.2. The reconstituted lyophilizate preferably has a pH of between 5-6.5 e.g. between 5.8-6.2, or 5.9-6.1, or a pH of 6. A liquid material for reconstitution can include an adjuvant e.g. an aluminium salt adjuvant. Aqueous suspensions of adjuvants (optionally including buffers, such as a histidine buffer) are useful for simultaneously reconstituting and adsorbing lyophilized polypeptides. In other embodiments the liquid material is adjuvant-free. Typically the lyophilizate does not include an insoluble metal salt adjuvant.

The invention also provides a lyophilizate which comprises EDTA and at least one antigen.

Immunogenic Compositions and Medicaments

Immunogenic compositions of the invention may be useful as vaccines. Vaccines according to the invention may either be prophylactic (i.e. to prevent infection) or therapeutic (i.e. to treat infection), but will typically be prophylactic.

Compositions may thus be pharmaceutically acceptable. They will usually include components in addition to the antigens e.g. they typically include one or more pharmaceutical carrier(s) and/or excipient(s). A thorough discussion of such components is available in Reference 38.

Compositions will generally be administered to a mammal in aqueous form. Prior to administration, however, the composition may have been in a non-aqueous form. For instance, although some immunogenic compositions are manufactured in aqueous form, then filled and distributed and administered also in aqueous form, other immunogenic compositions are lyophilized during manufacture and are reconstituted into an aqueous form at the time of use. Thus a composition of the invention may be dried, such as a lyophilized formulation.

Where a composition of the invention includes more than one polypeptide, the mass of each different polypeptide can be the same or different. Ideally they are present at substantially equal masses i.e. the mass of each of them is within ±5% of the mean mass of all the polypeptides. In embodiments where two antigens are present as a hybrid polypeptide, the hybrid is considered as a single polypeptide for this purpose. The factors that can influence the amount of the polypeptide to be included in a multivalent formulation include the amount of polypeptide sufficient to elicit an immune response and the amount that would cause aggregation (with itself or with other polypeptide) or influence the stability of the other polypeptide. Typical masses of a polypeptide in an immunogenic composition are between 1-100 μg.

The composition may include preservatives such as thiomersal or 2-phenoxyethanol. It is preferred, however, that the immunogenic compositions should be substantially free from (i.e. less than 5 μg/ml) mercurial material e.g. thiomersal-free. Compositions containing no mercury are more preferred. Preservative-free compositions are particularly preferred.

To improve thermal stability, a composition may include a temperature protective agent. Further details of such agents are provided below. To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminium hydroxide adjuvant); or a citrate buffer. Buffers will typically be included in the 5-20 mM range. The buffer is preferably 10 mM potassium phosphate.

The pH of the compositions are preferably between about 5 and about 8, and more preferably between about 5.5 and about 6.5, and most preferably at about 6.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Immunogenic compositions of the invention may also comprise one or more immunoregulatory agents. Preferably, one or more of the immunoregulatory agents include one or more adjuvants. The adjuvants may include a TH1 adjuvant and/or a TH2 adjuvant, further discussed below. Thus the immunogenic compositions may further comprise an adjuvant, such as an aluminium salt adjuvant (for example, one or more antigens may be adsorbed to aluminium salt). More generally, adjuvants which may be used in compositions of the invention include, but are not limited to, those already listed in reference 5. These include mineral-containing adjuvants and oil-in-water emulsions.

Mineral-Containing Adjuvants

Mineral containing adjuvants include mineral salts such as aluminium salts and calcium salts (or mixtures thereof). Preferably, the composition contains an aluminium salt adjuvant. Aluminium salts include hydroxides, phosphates, etc., with the salts taking any suitable form (e.g. gel, crystalline, amorphous, etc.). Calcium salts include calcium phosphate (e.g. the “CAP” particles disclosed in Ref 13). Adsorption to these salts is preferred (e.g. all antigens may be adsorbed). The mineral containing compositions may also be formulated as a particle of metal salt [14].

The adjuvants known as aluminium hydroxide and aluminium phosphate may be used. These names are conventional, but are used for convenience only, as neither is a precise description of the actual chemical compound which is present (e.g. see chapter 9 of Reference 15)). The invention can use any of the “hydroxide” or “phosphate” adjuvants that are in general use as adjuvants. The adjuvants known as “aluminium hydroxide” are typically aluminium oxyhydroxide salts, which are usually at least partially crystalline. The adjuvants known as “aluminium phosphate” are typically aluminium hydroxyphosphates, often also containing a small amount of sulphate (i.e. aluminium hydroxyphosphate sulphate). They may be obtained by precipitation, and the reaction conditions and concentrations during precipitation influence the degree of substitution of phosphate for hydroxyl in the salt.

A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

Aluminium phosphate adjuvants generally have a PO₄/Al molar ratio between 0.3 and 1.2, preferably between 0.8 and 1.2, and more preferably 0.95+0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.1-10 μm (e.g. about 0.1-5 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

The preferred aluminium salt adjuvant is an aluminium hydroxide adjuvant.

The invention can use a mixture of both an aluminium hydroxide and an aluminium phosphate. In this case there may be more aluminium phosphate than hydroxide e.g. a weight ratio of at least 2:1 e.g. ≧5:1, ≧6:1, ≧7:1, ≧8:1, ≧9:1, etc.

The concentration of Al⁺⁺⁺ in a composition for administration to a patient is preferably less than 10 mg/ml e.g. ≦5 mg/ml, ≦4 mg/ml, ≦3 mg/ml, ≦2 mg/ml, ≦1 mg/ml, etc. A preferred range is between 0.3 and 1 mg/ml. A maximum of 0.85 mg/dose is preferred.

A mineral salt can usefully have a TLR agonist, such as a TLR7 agonist, adsorbed to it (e.g. see Ref 16). The adsorbed TLR7 agonist is usefully a compound of formula (K) as described above.

Oil & Water Emulsions

Oil emulsion compositions suitable for use as adjuvants in the invention include oil-in-water emulsions such as MF59 (Chapter 10 of Ref 15; see also Ref 17) and AS03. Complete Freund's adjuvant (CFA) and incomplete Freund's adjuvant (IFA) may also be used.

Various oil-in-water emulsion adjuvants are known, and they typically include at least one oil and at least one surfactant, with the oil(s) and surfactant(s) being biodegradable (metabolisable) and biocompatible. The oil droplets in the emulsion are generally less than 5 μm in diameter, and ideally have a sub-micron diameter, with these small sizes being achieved with a microfluidiser to provide stable emulsions. Droplets with a size less than 220 nm are preferred as they can be subjected to filter sterilization.

The emulsion can comprise oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalane, the saturated analog to squalene, is also a preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAXTM tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate), Span 85 (sorbitan trioleate), lecithin and Triton X-100.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Preferred emulsion adjuvants have an average droplets size of ≦1 μm e.g. ≦750 nm, ≦500 nm, ≦400 nm, ≦300 nm, ≦250 nm, ≦220 nm, ≦200 nm, or smaller. These droplet sizes can conveniently be achieved by techniques such as microfluidisation.

Specific oil-in-water emulsion adjuvants useful with the invention include, but are not limited to:
A submicron emulsion of squalene, polysorbate 80, and sorbitan trioleate. These three components can be present at a volume ratio of 10:1:1 or a weight ratio of 39:47:47. The composition of the emulsion by volume can be about 5% squalene, about 0.5% polysorbate 80 and about 0.5% sorbitan trioleate. In weight terms, these ratios become 4.3% squalene, 0.5% polysorbate 80 and 0.48% sorbitan trioleate. This adjuvant is known as ‘MF59’ [18-20], as described in more detail in Chapter 10 of Ref 21 and chapter 12 of Ref 22. The MF59 emulsion advantageously includes citrate ions e.g. 10 mM sodium citrate buffer.
An emulsion of squalene, a tocopherol, and polysorbate 80. The emulsion may include phosphate buffered saline. It may also include Span 85 (e.g. at 1%) and/or lecithin. These emulsions may have from 2 to 10% squalene, from 2 to 10% tocopherol and from 0.3 to 3% polysorbate 80, and the weight ratio of squalene:tocopherol is preferably <1 as this provides a more stable emulsion. Squalene and polysorbate 80 may be present volume ratio of about 5:2 or at a weight ratio of about 11:5. Thus the three components (squalene, tocopherol, polysorbate 80) may be present at a weight ratio of 1068:1186:485 or around 55:61:25. One such emulsion (‘AS03’) can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm. The emulsion may also include a 3-de-O-acylated monophosphoryl lipid A (3d-MPL). Another useful emulsion of this type may comprise, per human dose, 0.5-10 mg squalene, 0.5-11 mg tocopherol, and 0.1-4 mg polysorbate 80 [23] e.g. in the ratios discussed above.
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 m/ml polysorbate 80, 110 m/ml Triton X-100 and 100 m/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [24] (0.05-1% Thr-MDP, 5% squalane, 2.5% Pluronic L121 and 0.2% polysorbate 80). It can also be used without the Thr-MDP, as in the “AF” adjuvant [25] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [26]. The emulsion may also include one or more of: alditol; a cryoprotective agent (e.g. a sugar, such as dodecylmaltoside and/or sucrose); and/or an alkylpolyglycoside. The emulsion may include a TLR4 agonist [27]. Such emulsions may be lyophilized.
An emulsion of squalene, poloxamer 105 and Abil-Care [28]. The final concentration (weight) of these components in adjuvanted vaccines are 5% squalene, 4% poloxamer 105 (pluronic polyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in Reference 29, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in Reference 30, produced by addition of aliphatic amine to desacylsaponin via the carboxyl group of glucuronic acid), dimethyidioctadecylammonium bromide and/or N,N-dioctadecyl-N,N-bis (2-hydroxyethyl)propanediamine.
An emulsion in which a saponin (e.g. QuilA or QS21) and a sterol (e.g. a cholesterol) are associated as helical micelles [31].
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [32].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [32].

In some embodiments an emulsion may be mixed with antigen extemporaneously, at the time of delivery, and thus the adjuvant and antigen may be kept separately in a packaged or distributed composition, ready for final formulation at the time of use. In other embodiments an emulsion is mixed with antigen during manufacture, and thus the composition is packaged in a liquid adjuvanted form. The antigen will generally be in an aqueous form, such that the composition is finally prepared by mixing two liquids. The volume ratio of the two liquids for mixing can vary (e.g. between 5:1 and 1:5) but is generally about 1:1. Where concentrations of components are given in the above descriptions of specific emulsions, these concentrations are typically for an undiluted composition, and the concentration after mixing with an antigen solution will thus decrease.

Where a composition includes a tocopherol, any of the α, β, γ, δ, ε or ξ tocopherols can be used, but α-tocopherols are preferred. The tocopherol can take several forms e.g. different salts and/or isomers. Salts include organic salts, such as succinate, acetate, nicotinate, etc. D-α-tocopherol and DL-α-tocopherol can both be used. Tocopherols are advantageously included in compositions for use in elderly patients (e.g. aged 60 years or older) because vitamin E has been reported to have a positive effect on the immune response in this patient group [33]. They also have antioxidant properties that may help to stabilize the emulsions [34]. A preferred α-tocopherol is DL-α-tocopherol, and the preferred salt of this tocopherol is the succinate.

The use of an aluminium hydroxide and/or aluminium phosphate adjuvant is particularly preferred, and antigens are generally adsorbed to these salts.

Compositions of the invention may elicit both a cell mediated immune response as well as a humoral immune response. This immune response will preferably induce long lasting (e.g. neutralising) antibodies and a cell mediated immunity that can quickly respond upon exposure to S. aureus.

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

S. aureus infections can affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilized composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilized antigens.

Where a composition is to be prepared extemporaneously prior to use (e.g. where a component is presented in lyophilized form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Where more than one antigen is included in a composition then two antigens may be present at the same dose as each other or at different doses.

As mentioned above, a composition may include a temperature protective agent, and this component may be particularly useful in adjuvanted compositions (particularly those containing a mineral adjuvant, such as an aluminium salt). As described in Reference 35, a liquid temperature protective agent may be added to an aqueous vaccine composition to lower its freezing point e.g. to reduce the freezing point to below 0° C. Thus the composition can be stored below 0° C., but above its freezing point, to inhibit thermal breakdown. The temperature protective agent also permits freezing of the composition while protecting mineral salt adjuvants against agglomeration or sedimentation after freezing and thawing, and may also protect the composition at elevated temperatures e.g. above 40° C. A starting aqueous vaccine and the liquid temperature protective agent may be mixed such that the liquid temperature protective agent forms from 1-80% by volume of the final mixture. Suitable temperature protective agents should be safe for human administration, readily miscible/soluble in water, and should not damage other components (e.g. antigen and adjuvant) in the composition. Examples include glycerin, propylene glycol, and/or polyethylene glycol (PEG). Suitable PEGs may have an average molecular weight ranging from 200-20,000 Da. In a preferred embodiment, the polyethylene glycol can have an average molecular weight of about 300 Da (TEG-300′).

Methods of Treatment, and Administration of the Vaccine

The invention also provides a method for raising an immune response in a mammal comprising the step of administering a composition of the invention to the mammal The immune response is preferably protective and preferably involves antibodies and/or cell-mediated immunity The method may raise a booster response.

At least some of the antibodies raised in response to polypeptides which are administered in accordance with the invention should be protective.

The invention also provides the use of a variant form of a Sta011 antigen, provided that the variant does not contain any free thiol group, in the manufacture of a medicament for raising an immune response in a mammal. It may also involve the use of an adjuvant.

By raising an immune response in the mammal by these uses and methods, the mammal can be protected against S. aureus infection, including a nosocomial infection. More particularly, the mammal may be protected against a skin infection, pneumonia, meningitis, osteomyelitis endocarditis, toxic shock syndrome, and/or septicaemia.

The invention also provides a kit comprising a first component and a second component wherein neither the first component nor the second component is a composition of the invention as described above, but wherein the first component and the second component can be combined to provide a composition of the invention as described above. The kit may further include a third component comprising one or more of the following: instructions, syringe or other delivery device, adjuvant, or pharmaceutically acceptable formulating solution.

The invention also provides a delivery device pre-filled with an immunogenic composition of the invention.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc. Other mammals which can usefully be immunised according to the invention are cows, dogs, horses, and pigs.

One way of checking efficacy of therapeutic treatment involves monitoring S. aureus infection after administration of the compositions of the invention. One way of checking efficacy of prophylactic treatment involves monitoring immune responses, systemically (such as monitoring the level of IgG1 and IgG2a production) and/or mucosally (such as monitoring the level of IgA production), against the antigens in the compositions of the invention after administration of the composition. Typically, antigen-specific serum antibody responses are determined post-immunisation but pre-challenge whereas antigen-specific mucosal antibody responses are determined post-immunisation and post-challenge.

Another way of assessing the immunogenicity of the compositions of the present invention is to express the proteins recombinantly for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within antigens.

The efficacy of immunogenic compositions can also be determined in vivo by challenging animal models of S. aureus infection, e.g., guinea pigs or mice, with the immunogenic compositions. In particular, there are three useful animal models for the study of S. aureus infectious disease, namely: (i) the murine abscess model [36], (ii) the murine lethal infection model [36] and (iii) the murine pneumonia model [37]. The abscess model looks at abscesses in mouse kidneys after intravenous challenge. The lethal infection model looks at the number of mice which survive after being infected by a normally-lethal dose of S. aureus by the intravenous or intraperitoneal route. The pneumonia model also looks at the survival rate, but uses intranasal infection. A useful immunogenic composition may be effective in one or more of these models. For instance, for some clinical situations it may be desirable to protect against pneumonia, without needing to prevent hematic spread or to promote opsonisation; in other situations the main desire may be to prevent hematic spread. Different antigens, and different antigen combinations, may contribute to different aspects of an effective immunogenic composition.

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal or transcutaneous, intranasal, ocular, aural, pulmonary or other mucosal administration.

The invention may be used to elicit systemic and/or mucosal immunity, preferably to elicit an enhanced systemic and/or mucosal immunity

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines prepared according to the invention may be used to treat both children and adults. Thus a human patient may be less than 1 year old, 1-5 years old, 5-15 years old, 15-55 years old, or at least 55 years old. Preferred patients for receiving the vaccines are the elderly (e.g. ≧50 years old, ≧60 years old, and preferably ≧65 years), the young (e.g. ≦5 years old), hospitalised patients, healthcare workers, armed service and military personnel, pregnant women, the chronically ill, or immunodeficient patients. The vaccines are not suitable solely for these groups, however, and may be used more generally in a population.

Vaccines produced by the invention may be administered to patients at substantially the same time as (e.g. during the same medical consultation or visit to a healthcare professional or vaccination centre) other vaccines e.g. at substantially the same time as an influenza vaccine, a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, etc. Further non-staphylococcal vaccines suitable for co-administration may include one or more antigens listed on pages 33-46 of Reference 11.

General

The practice of the present invention will employ, unless otherwise indicated, conventional methods of chemistry, biochemistry, molecular biology, immunology and pharmacology, within the skill of the art. Such techniques are explained fully in the literature. See, e.g., References 38-45, etc.

“GI” numbering is used above. A GI number, or “GenInfo Identifier”, is a series of digits assigned consecutively to each sequence record processed by NCBI when sequences are added to its databases. The GI number bears no resemblance to the accession number of the sequence record. When a sequence is updated (e.g. for correction, or to add more annotation or information) then it receives a new GI number. Thus the sequence associated with a given GI number is never changed.

Where the invention concerns an “epitope”, this epitope may be a B-cell epitope and/or a T-cell epitope. Such epitopes can be identified empirically (e.g. using PEPSCAN [46,47] or similar methods), or they can be predicted (e.g. using the Jameson-Wolf antigenic index [48], matrix-based approaches [49], MAPITOPE [50], TEPITOPE [51,52], neural networks [53], OptiMer & EpiMer [54, 55], ADEPT [56], Tsites [57], hydrophilicity [58], antigenic index [59] or the methods disclosed in References 60-64, etc.). Epitopes are the parts of an antigen that are recognised by and bind to the antigen binding sites of antibodies or T-cell receptors, and they may also be referred to as “antigenic determinants”.

Where an antigen “domain” is omitted, this may involve omission of a signal peptide, of a cytoplasmic domain, of a transmembrane domain, of an extracellular domain, etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of Ref 65. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in Ref 66. The percentage sequence identity between two sequences of different lengths is preferably calculated over the length of the longer sequence.

Phosphorous-containing adjuvants used with the invention may exist in a number of protonated and deprotonated forms depending on the pH of the surrounding environment, for example the pH of the solvent in which they are dissolved. Therefore, although a particular form may be illustrated, it is intended that these illustrations are merely representative and not limiting to a specific protonated or deprotonated form. For example, in the case of a phosphate group, this has been illustrated as —OP(O)(OH)₂ but the definition includes the protonated forms [OP(O)(OH₂)(OH)]⁺ and —[OP(O)(OH)₂]²⁺ that may exist in acidic conditions and the deprotonated forms —[OP(O)(OH)(O)]⁻ and [OP(O)(O)₂]²⁻ that may exist in basic conditions.

Compounds can exist as pharmaceutically acceptable salts. Thus, compounds (e.g. adjuvants) may be used in the form of their pharmaceutically acceptable salts i.e. physiologically or toxicologically tolerable salt (which includes, when appropriate, pharmaceutically acceptable base addition salts and pharmaceutically acceptable acid addition salts).

The word “substantially” does not exclude “completely” e.g. a composition which is “substantially free” from Y may be completely free from Y. Where necessary, the word “substantially” may be omitted from the definition of the invention.

### BRIEF DESCRIPTION OF DRAWINGS

FIG. 1 shows the normalised melting curves of the cysteine-containing and cysteine-deficient Sta011 antigens.

FIG. 2 shows the size exclusion chromatography profile of Sta011 Cys(−) antigen after 4 weeks storage at 25° C. The chromatogram of Sta011 Cys(−) antigen without having undergone storage condition is also shown as a control.

FIG. 3 shows the size exclusion chromatography profile of Sta011 Cys(−) antigen with and without adjuvant (aluminium hydroxide) after 4 weeks storage at 37° C. The chromatogram of Sta011 Cys(−) antigen without having undergone storage condition is also shown as a control. * indicates additional peak of Sta011 Cys(−) antigen without adjuvant stored after 4 weeks at 37° C.

FIG. 4 shows anti-Sta011 antibody titres in CD1 mice which have been immunized with various vaccines: (A) a combination based on Reference 5 that contains Sta011 Cys(+) antigen, (B) a combination based on Reference 5 that contains Sta011 Cys(−) antigen, (D) a monovalent Sta011 Cys(−) vaccine, and (H) a monovalent Sta011 Cys(+) vaccine. (C) and (G) are Sta006 controls, (E) and (I) are EsxAB controls, (F) and (J) are Hla controls.

### MODES FOR CARRYING OUT THE INVENTION

Thermal Denaturation Assay

The Sta011 Cys(+) antigen used in the experiments described below is represented by SEQ ID NO: 6, and the Sta011 Cys(−) antigen is represented by SEQ ID NO: 11. Both antigens were recombinant proteins purified from E. coli.

Thermal stability of the Sta011 cysteine-containing Cys(+) antigen was compared the Sta011 cysteine-deficient Cys(−) antigen by Differential Scanning Fluorimetry (DSF). Samples containing antigen (10 μM in PBS) were heated under controlled conditions with a ramp rate of 1° C./min in Strategen Mx3000p Real Time PCR instrument. The dye SyproOrange 5× was used, and the changes in fluorescence were monitored. Assays were performed over a temperature range of 10-100° C.

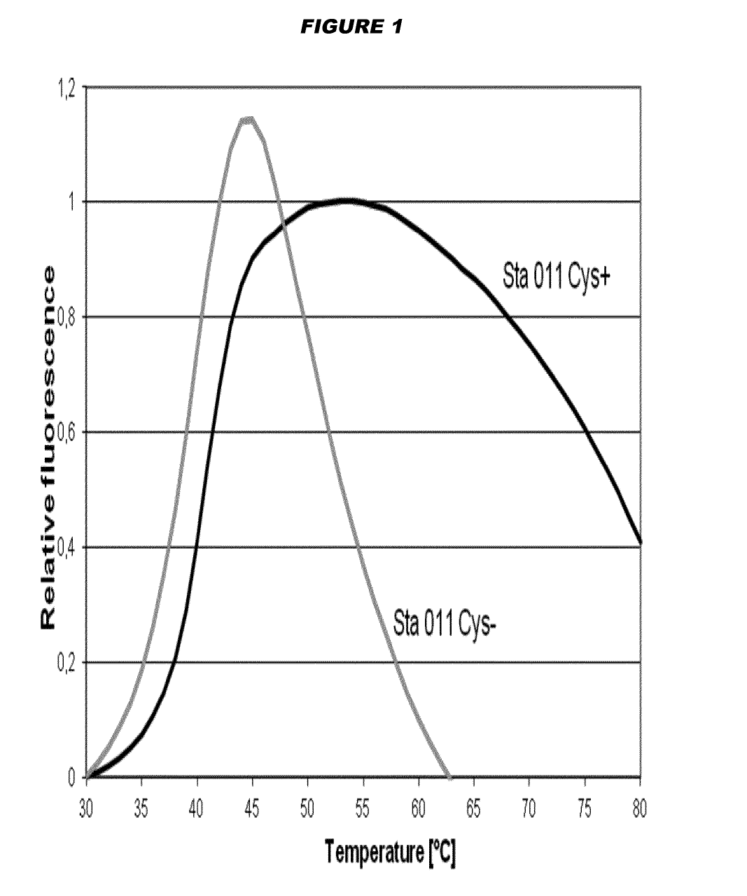

FIG. 1 reports the melting curves of the antigens tested. It is shown that the peak for the Cys(−) antigen is shifted slightly to the top and left compared to the Cys(+) antigen. Melting temperatures (Tm) were determined by fitting the first derivative of the experimental curve. The Tm of the Sta011 Cys(+) antigen was 40.6° C., and the Tm of the Sta011 Cys(−) antigen was 39.12° C.

Thus, the thermal stability profile of the Sta011 Cys(−) antigen is comparable to the Sta011 Cys(+) antigen. Modifying the antigen by deleting or replacing the cysteine residue does not have a significant impact on the thermal stability of the Sta011 antigen.

Purification Process

The purification steps for Sta011 are explained below.
1. Lysis and clarification—cell lysis and clarification adding a flocculating agent (PEI) that reduces DNA, endotoxins and proteic impurities.
2. CaptoQ chromatography—removal of HCP and other impurities and dimerization of Cys(+) antigen.
3. cHT chromatography—removal of HCP and other impurities and separation of monomer from dimer
4. Final 10 kDa diafiltration—diafiltration in final buffer.

For purifying Sta011 Cys(−), the CaptoQ chromatography step was simplified as the antigen can be purified as a monomer. The cHT chromatography step was also simplified because it was no longer necessary to separate the monomer from the dimer

Purity and yield of the antigens obtained from the process explained above were determined, and the results are shown in Table 1. Purity is determined using detector PDA 214 nm. Yield is calculated by: total proteins (mBCA content (mg/ml))×purity (RPC (%) 214 nm).

TABLE 1
Purity and yield of the Cys(−) and Cys(+) antigens.

| Antigen | Cys? | RP purity (%) | Yield (g/L ferm) |
|---|---|---|---|
| Sta011 | Cys(−) | 90.1 | 0.091 |
| | | | Biomass of fermentation recovered |
| | | | was about 79% (theoretical yield |
| | | | without slurry from centrifugation |
| | | | loss was: 0.110 g/L ferm) |
| | Cys(+) | 95.8 | 0.012 |

The purified Sta011 Cys(−) antigen had comparable purity and yield to the Sta011 Cys(+) antigen. The analytical panel conformed to in-house specification limits. Removal of cysteine allowed higher flexibility in the purification process, and hence facilitates easier characterization during vaccine production. The purification process can be further optimised in order to improve purity and yield.

Thermal Stability Evaluation

The stability of Sta011 Cys(−) antigen in a vaccine combination based on the disclosure of Reference 5 was investigated. The antigen was present at a concentration of 72 μg/mL. The vaccine combination was tested with or without adjuvant. The vaccine combination was exposed to temperatures: 2-8° C., 15° C., 25° C., 37° C. for 0 to 4 weeks. The highest temperature tested (37° C.) was below the Tm of the Sta011 Cys(−) antigen (about 40° C.). Hence, protein instability driven by the protein unfolding was not an influencing factor in this experiment.

The samples were analysed using RP-HPLC, and the pH and osmolality were also analysed (3 determinations on 3 different vials at each temperature and timepoint). For desorption, the samples were treated with 300 mM KH2PO4 pH 6.8 overnight at 25° C. The same conditions were applied for sample treatment at all time points (assumption: no influence of formulation aging).

It was observed that the Sta011 Cys(−) antigen was completely adsorbed onto Alum with adsorption>96%. The osmolality and pH remained constant over time and within acceptable range. The Sta011 Cys(−) antigen was stable when stored for four weeks at 2-8° C., 15° C. and 25° C. However, when stored for 4 weeks at 37° C., there was 20-30% loss of antigen.

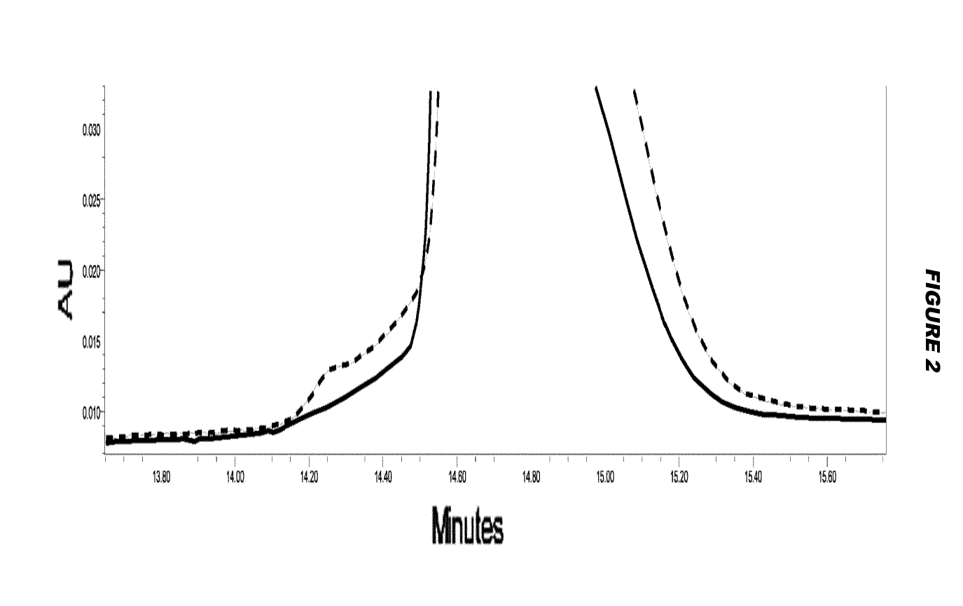

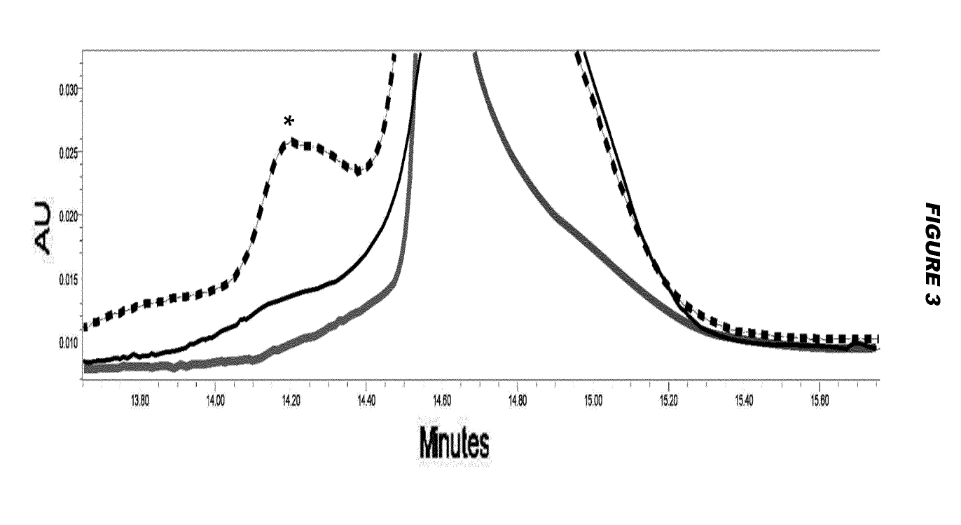

No additional peaks (e.g. degradation products) were seen in the HPLC profiles of the desorbed samples at any condition tested, except at T=37° C. FIG. 2 shows that Sta011 Cys(−) antigen (non-adjuvanted) stored after 4 weeks at 25° C. is as stable as the control. FIG. 3 shows that Sta011 Cys(−) antigen (non-adjuvanted) stored after 4 weeks at 37° C. is relatively less stable compared to the control. There is an additional peak (*), suggesting the presence of degradation products, in the RP-HPLC profile of Sta011 Cys(−) antigen. The area of the additional peak over total area is 4.2%.

FIG. 3 also shows that Sta011 Cys(−) antigen adjuvanted with aluminium hydroxide is more stable than the non-adjuvanted Sta011 Cys(−) antigen after 4 weeks storage at 37° C.

Hence, the Sta011 Cys(−) antigen was stable for up to 4 weeks at 2-8° C., 15° C. and 25° C. When stored for 4 weeks at 37° C., Sta011 Cys(−) was less stable, but its stability was improved in the presence of adjuvant, e.g. aluminium hydroxide.

Immunogenicity Studies in Mice

Immunogenicity of the Sta011 Cys(+) antigen was compared with the Sta011 Cys(−) antigen. The antigens were adjuvanted with aluminium hydroxide, and were used as a monovalent vaccine or in a combination vaccine based on the disclosure of Reference 5. Each vaccine contains 30 μg of each antigen and aluminium hydroxide at 2 mg/ml.

Sixteen CD1 mice (five week old) were immunized subcutaneously three times (at t=0, 14 and 28 days). Animals were bled immediately prior to the first immunization, and 13, 27 and 42 days following the first immunization. The sera were examined for IgG antibodies directed against the purified proteins using the Luminex technology. The assay read-out is a measure of fluorescence intensity expressed as arbitrary Relative Luminex Units (RLU/mL).

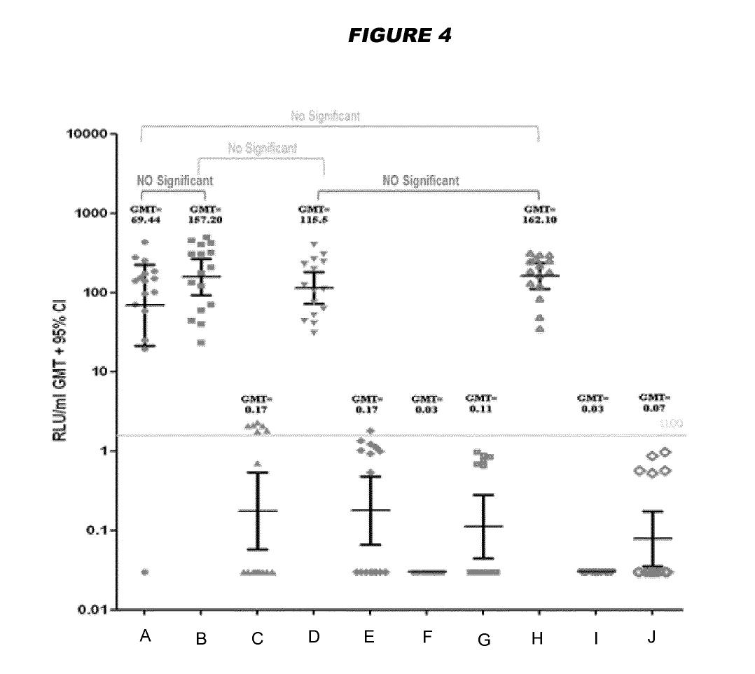

FIG. 4 reports antibody titres of mice 42 days following immunization. Anti-Sta011 antibodies were specifically elicited by vaccines containing the Sta011 Cys(−) and Cys(+) antigens. There is no significant difference between the antibodies elicited by the vaccines (monovalent and combination) containing the Sta011 Cys(−) antigen and the Cys(+) antigen.

Table 2 shows the GMT values of the immunogenicity studies. There is no significant difference between the vaccines (monovalent and combination) containing the Sta011 Cys(−) antigen and the Cys(+) antigen at 42 days following immunization.

TABLE 2
GMT values after third immunization (t = 42 days).

| Vaccine | GMT |
|---|---|
| Monovalent Sta011 Cys(+) | 162.10 |
| Monovalent Sta011 Cys(−) | 115.50 |
| Combination containing | 69.44 |
| Sta011 Cys(+) | |
| Combination containing | 157.20 |
| Sta011 Cys(−) | |

It will be understood that the invention is described above by way of example only and modifications may be made whilst remaining within the scope and spirit of the invention.

### REFERENCES

[1] Harro et al. (2010) Clin Vaccine Immunol 17:1868-74.

[2] Kuklin et al. (2006) Infect Immun. 74(4):2215-23.

[3] Sheridan (2009) Nature Biotechnology 27:499-501.

[4] Merck and Intercell AG Announce Termination of Phase II/III Clinical Trial of Investigational Staphylococcus aureus Vaccine, V710—Merck Research and Development News—8 Jun. 2011

[5] WO2010/119343.

[6] U.S. provisional application 61/580,191.

[7] Protective Groups in organic synthesis, Third Edition, Theodora W Greene, Peter G M Wuts, (1999) John Wiley, Chapter 6 Protection for the Thiol Group.

[8] Kuroda et al. (2001) Lancet 357:1225-1240.

[9] Needleman & Wunsch (1970) J. Mol. Biol. 48, 443-453.

[10] Rice et al. (2000) Trends Genet 16:276-277.

[11] WO2008/019162.

[12] WO2011/027222.

[13] U.S. Pat. No. 6,355,271.

[14] WO00/23105.

[15] Vaccine Design . . . (1995) eds. Powell & Newman. ISBN: 030644867X. Plenum.

[16] WO2011/027222.

[17] WO90/14837.

[18] WO90/14837.

[19] Podda & Del Giudice (2003) Expert Rev Vaccines 2:197-203.

[20] Podda (2001) Vaccine 19: 2673-2680.

[21] Vaccine Design: The Subunit and Adjuvant Approach (eds. Powell & Newman) Plenum Press 1995 (ISBN 0-306-44867-X).

[22] Vaccine Adjuvants: Preparation Methods and Research Protocols (Volume 42 of Methods in Molecular Medicine series). ISBN: 1-59259-083-7. Ed. O'Hagan.

[23] WO2008/043774.

[24] Allison & Byars (1992) Res Immunol 143:519-25.

[25] Hariharan et al. (1995) Cancer Res 55:3486-9.

[26] US-2007/014805.

[27] US-2007/0191314.

[28] Suli et al. (2004) Vaccine 22(25-26):3464-9.

[29] WO95/11700.

[30] U.S. Pat. No. 6,080,725.

[31] WO2005/097181.

[32] WO2006/113373.

[33] Han et al. (2005) Impact of Vitamin E on Immune Function and Infectious Diseases in the Aged at Nutrition, Immune functions and Health EuroConference, Paris, 9-10 Jun. 2005.

[34] U.S. Pat. No. 6,630,161.

[35] WO2006/110603.

[36] Stranger-Jones et al. (2006) PNAS USA 103:16942-7.

[37] Wardenburg et al. (2007) Infect Immun 75:1040-4.

[38] Gennaro (2000) Remington: The Science and Practice of Pharmacy. 20th edition, ISBN: 0683306472.

[39] Methods In Enzymology (S. Colowick and N. Kaplan, eds., Academic Press, Inc.)

[40] Handbook of Experimental Immunology, Vols. I-IV (D. M. Weir and C. C. Blackwell, eds, 1986, Blackwell Scientific Publications)

[41] Sambrook et al. (2001) Molecular Cloning: A Laboratory Manual, 3rd edition (Cold Spring Harbor Laboratory Press).

[42] Handbook of Surface and Colloidal Chemistry (Birdi, K. S. ed., CRC Press, 1997)

[43] Ausubel et al. (eds) (2002) Short protocols in molecular biology, 5th edition (Current Protocols).

[44] Molecular Biology Techniques: An Intensive Laboratory Course, (Ream et al., eds., 1998, Academic Press)

[45] PCR (Introduction to Biotechniques Series), 2nd ed. (Newton & Graham eds., 1997, Springer Verlag)

[46] Geysen et al. (1984) PNAS USA 81:3998-4002.

[47] Carter (1994) Methods Mol Biol 36:207-23.

[48] Jameson, B A et al. 1988, CABIOS 4(1):181-186.

[49] Raddrizzani & Hammer (2000) Brief Bioinform 1(2):179-89.

[50] Bublil et al. (2007) Proteins 68(1):294-304.

[51] De Lalla et al. (1999) J. Immunol. 163:1725-29.

[52] Kwok et al. (2001) Trends Immunol 22:583-88.

[53] Brusic et al. (1998) Bioinformatics 14(2):121-30

[54] Meister et al. (1995) Vaccine 13(6):581-91.

[55] Roberts et al. (1996) AIDS Res Hum Retroviruses 12(7):593-610.

[56] Maksyutov & Zagrebelnaya (1993) Comput Appl Biosci 9(3):291-7.

[57] Feller & de la Cruz (1991) Nature 349(6311):720-1.

[58] Hopp (1993) Peptide Research 6:183-190.

[59] Welling et al. (1985) FEBS Lett. 188:215-218.

[60] Davenport et al. (1995) Immunogenetics 42:392-297.

[61] Tsurui & Takahashi (2007) J Pharmacol Sci. 105(4):299-316.

[62] Tong et al. (2007) Brief Bioinform. 8(2):96-108.

[63] Schirle et al. (2001) J Immunol Methods. 257(1-2):1-16.

[64] Chen et al. (2007) Amino Acids 33(3):423-8.

[65] Current Protocols in Molecular Biology (F. M. Ausubel et al., eds., 1987) Supplement 30

[66] Smith & Waterman (1981) Adv. Appl. Math. 2: 482-489.

1. A polypeptide comprising an amino acid sequence having at least 80% identity to SEQ ID NO: 7, wherein the polypeptide has no free thiol group and can elicit antibodies which recognise SEQ ID NO: 5.

2. A hybrid protein comprising the polypeptide of claim 1.

3. A nucleic acid molecule comprising a nucleotide sequence encoding the polypeptide of claim 2.

4. A vector comprising the nucleic acid molecule of claim 3.

5. A host cell comprising the vector of claim 4.

6. A method for preparing the polypeptide of claim 1, comprising the steps of:
culturing the host cell of claim 5 under conditions whereby said polypeptide is expressed; and,
recovering said polypeptide.

7. An immunogenic composition comprising the polypeptide of claim 1.

8. The immunogenic composition of claim 7, further comprising one or more conjugates of (i) a S. aureus exopolysaccharide and (ii) a carrier protein.

9. The immunogenic composition of claim 7, further comprising one or more conjugates of (i) a S. aureus capsular polysaccharide and (ii) a carrier protein.

10. The immunogenic composition of claim 7, further comprising an adjuvant, a saccharide, or combination thereof.

11. The immunogenic composition of claim 7, further comprising a stabilizing additive.

12. The immunogenic composition of claim 7, in a lyophilized form.

13. The immunogenic composition of claim 7, in an aqueous form.

14. A method for preparing the immunogenic composition of claim 13, the method comprising the step of:
reconstituting the immunogenic composition of claim 12 with aqueous material, so as to form the aqueous form.

15. A vaccine comprising the polypeptide of claim 1.

16. A method for raising an immune response in a mammal, the method comprising the step of:
administering to the mammal the polypeptide of claim 1, in an amount effective to raise an immune response.

17. The method of claim 16, wherein the amount effective to raise an immune response is an amount effective to treat an S. aureus infection in the mammal.

18. The method of claim 16, wherein the amount effective to raise an immune response is an amount effective to prevent an S. aureus infection in the mammal.


SEQUENCE LISTING (15 sequences)

SEQ ID NO: 1 - protein, 256 aa, Staphylococcus aureus
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 2 - protein, 256 aa, Artificial Sequence
  Staphylococcus aureus protein variant
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKLKKEIENFKFFVQYGDFKNIKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 3 - protein, 256 aa, Artificial Sequence
  Staphylococcus aureus protein variant
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKVKKEIENFKFFVQYGDFKNIKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 4 - protein, 256 aa, Artificial Sequence
  Staphylococcus aureus protein variant
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKLKKEIENFKFFVQYGDFKNVKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 5 - protein, 232 aa, Staphylococcus aureus
CGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 6 - protein, 234 aa, Artificial Sequence
  Staphylococcus aureus protein variant
MGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 7 - protein, 231 aa, Artificial Sequence
  Staphylococcus aureus protein variant
GIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 8 - protein, 232 aa, Artificial Sequence
  Staphylococcus aureus protein variant
XGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 9 - protein, 233 aa, Artificial Sequence
  Staphylococcus aureus protein variant
MGGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 10 - protein, 234 aa, Artificial Sequence
  Staphylococcus aureus protein variant
MGXGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 11 - protein, 234 aa, Artificial Sequence
  Staphylococcus aureus protein variant
MGSGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 12 - protein, 232 aa, Artificial Sequence
  Staphylococcus aureus protein variant
SGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 13 - protein, 232 aa, Artificial Sequence
  Staphylococcus aureus protein variant
CGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKLKKEIENFKFFVQYGDFKNIKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 14 - protein, 232 aa, Artificial Sequence
  Staphylococcus aureus protein variant
CGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKVKKEIENFKFFVQYGDFKNIKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 15 - protein, 232 aa, Artificial Sequence
  Staphylococcus aureus protein variant
CGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKLKKEIENFKFFVQYGDFKNVKNYKDGDISYNPEVPSYSAKYQLTNDDYNVKQLRKRYDIPTSKAPKLLLKGSGNLKGSSVGYKDIEFTFVEKKEENIYFSDSLDYKKSGDV